(19) United States
(12) Patent Application Publication
(10) Pub. No.: US 2004/0166079 A1
(43) Pub. Date: Aug. 26, 2004

(54) N-Lauroyl amino acid composition for slimming the human body

(76) Inventor: Christine Garcia, Castres (FR)

Correspondence Address: Linda K. Russell, Air Liquide, Suite 1800, 2700 Post Oak Blvd., Houston, TX, 77056, US

(21) Appl. No.: 10/783,569

(22) Filed: Feb. 20, 2004

(30) Foreign Application Priority Data
Feb. 21, 2003 (FR) 0302161

(51) Int. Cl. A61K 7/06; A61K 7/11

(52) U.S. Cl. 424/70.14

(57) ABSTRACT

Use of a compound of formula (I):

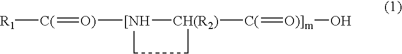

in which R₁ represents a hydrocarbon radical comprising 11 carbon atoms, R₂ represents the characterizing chain of an amino acid and m is between 1 and 50, or of a mixture of the said compounds of formula (I), as a slimming active agent. Nontherapeutic method of treatment using the said compound and use of the said compound for preparing a medicament with lipolytic activity, intended to induce slimming of the human body.

The subject of the present invention is a novel use of cosmetic active agents for slimming the human body.

Some of the fat in the human body is stored in the form of triglycerides, in cells of the fatty tissue of the dermis, called adipocytes. Slimming reflects a reduction in the fat stored in the adipocytes. This process requires a preliminary step which takes place inside these cells and which consists in hydrolysing the triglycerides to fatty acids and glycerol. This phenomenon is called lipolysis.

Most slimming cosmetic formulations currently marketed contain at least one compound possessing a lipolytic activity. The one most frequently used is caffeine, but theophylline is also known to possess such a property.

During their search for novel active agents with lipolytic activity which have better compatibility with the skin than those of the state of the art, the inventors demonstrated that certain N-acylated derivatives of amino acids known for their soothing property also had a lipolytic property which was more effective than that of caffeine.

Accordingly, according to a first aspect, the subject of the invention is the use of a compound of formula (I):

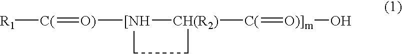

in which R₁ represents a linear or branched, saturated or unsaturated, aliphatic hydrocarbon radical comprising 11 carbon atoms, R₂ represents the characterizing chain of an amino acid and m is between 1 and 50, or of a mixture of the said compounds of formula (I), as a slimming active agent, in a composition containing a cosmetically acceptable medium.

The compound of formula (I) as defined above may be in the form of a free acid or in a partially or completely salified form. When the compound of formula (I) is in a salified form, it comprises in particular alkali metal salts such as the sodium, potassium or lithium salts, alkaline-earth metal salts such as the calcium, magnesium or strontium salts; an ammonium salt or a salt of an amino alcohol such as the (2-hydroxyethyl)-ammonium salt. It may also comprise metal salts such as divalent zinc or manganese salts, trivalent iron, lanthanum, cerium or aluminium salts. In general, the degree of salification of the compound of formula (I) as defined above will additionally depend on its p_{K}A and the salt concentration of the composition into which it is incorporated.

In the following disclosure, the expression compound of formula (I) is understood to mean a compound of formula (I) in free form or in a partially or completely salified form.

The expression “characterizing chain” used to define the radical R₂ denotes the nonfunctional principal chain of the amino acid considered.

Thus, for an amino acid represented by general formula (IIIa):

H₂N—CH(R₂)—C(═O)—OH (IIIa)

and for a cyclic amino acid represented by formula (IIIb):

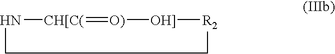

the characterizing chain will be the chain represented by R₂.

R₂ represents in particular the characterizing chain of an amino acid chosen from glycine, alanine, serine, aspartic acid, glutamic acid, valine, threonine, arginine, lysine, proline, leucine, phenylalanine, isoleucine, histidine, tyrosine, tryptophan, asparagine, glutamine, cysteine, cystine, methionine, hydroxyproline, hydroxylysine, sarcosine or ornithine.

The subject of the invention is mainly the use of a compound of formula (I) as defined above, in which, in at least one of the residues:

—HN—CH(R₂)—C(═O)— (IIIa)

or

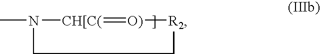

R₂ represents the characterizing chain of glycine, alanine, aspartic acid, glutamic acid or sarcosine.

The subject of the invention is more particularly the use of a compound of formula (I) as defined above, in which m is a decimal number between 1 and 10 and it is preferably less than 5.

According to a most particular aspect of the present invention, in formula (I) as defined above, m is less than or equal to 2 and is more particularly less than or equal to 1.4.

According to another most particular aspect of the present invention, in formula (I) as defined above, m is equal to 1.

According to another particular variant of the present invention, a single compound of formula (I) as defined above is used in the composition containing the cosmetically acceptable medium.

According to another particular variant of the present invention, a mixture of compounds of formula (I) as defined above is used.

The compounds of formulae (I) are generally obtained by N-acylation of compounds of formulae (IIIa) or (IIIb), as defined above, or their salts.

In the case of a mixture of compounds of formulae (I) it is for example obtained by N-acylation of the mixture of amino acids resulting from the total or partial hydrolysis of proteins of any origin.

These proteins may be of animal origin, such as for example collagen, elastin, fish flesh protein, fish gelatin, keratin or casein, of plant origin, such as cereal, flower or fruit proteins such as for example the proteins derived from soya bean, sunflower, oats, wheat, maize, barley, potato, lupin, field bean, sweet almond, kiwi, mango or apple; they may also be proteins obtained from Chorella (unicellular algae), pink algae, yeasts or silk.

This hydrolysis is carried out, for example, by heating to temperatures of between 60 and 130° C. a protein placed in an acidic or alkaline medium.

This hydrolysis may also be carried out enzymatically, with a protease, optionally coupled with a post-alkaline or post-acid hydrolysis. When m is greater than 1, R₂ represents a single chain or several chains characterizing different amino acids, depending on the protein hydrolysed and the degree of hydrolysis.

The aminograms of a few proteins of plant origin are presented in the following tables:

TABLE 1

Origin of the protein
(proportions of amino acids
expressed in wt %)

| | | Soya | | |
|---|---|---|---|---|
| | Oats | bean | Wheat | Sunflower |
| Glycine | 6.9 | 4.2 | 3.2 | 6.2 |
| Alanine | 5.9 | 4.2 | 2.6 | 4.8 |
| Serine | 5.6 | 5.1 | 1.7 | 5.1 |
| Aspartic acid | 16.2 | 11.7 | 3.4 | 10.6 |
| Glutamic acid | 28.3 | 19.1 | 37.9 | 23.6 |
| Valine | 2.9 | 5.0 | 4.2 | 4.8 |
| Threonine | 3.1 | 3.9 | 2.7 | 4.4 |
| Arginine | 6.6 | 7.8 | 3.7 | 8.4 |
| Lysine | 3.6 | 6.2 | 1.9 | 3.2 |
| Proline | 4.7 | 5.4 | 11.7 | 3.0 |
| Leucine | 6.4 | 8.1 | 7.1 | 6.4 |
| Phenylalanine | 1.4 | 5.0 | 5.4 | 4.3 |
| Isoleucine | 2.2 | 4.8 | 3.7 | 4.1 |
| Histidine | 1.7 | 2.6 | 2.4 | 2.0 |
| Tyrosine | 1.5 | 3.5 | 3.1 | 2.7 |
| Methionine | 1.2 | 1.2 | 1.6 | 1.8 |
| Cysteine/cystine | 1.9 | 1.5 | 1.9 | 1.9 |
| Tryptophan | — | 1.0 | 1.0 | 1.3 |

TABLE 2

Origin of the protein
(proportions of amino acids
expressed in wt %)

| | | | Field | |
|---|---|---|---|---|
| | Lupin | Potato | bean | Maize |
| Glycine | 0.9 | 4.8 | 4.0 | 2.4 |
| Alanine | 2.4 | 5.0 | 4.0 | 7.95 |
| Serine | 6.1 | 5.8 | 4.9 | 5.1 |
| Aspartic acid | 15.8 | 12.5 | 10.5 | 10.6 |
| Glutamic acid | 8.0 | 11.5 | 16.8 | 23.6 |
| Valine | 7.9 | 7.1 | 4.5 | 4.8 |
| Threonine | 8.1 | 6.1 | 3.6 | 4.4 |
| Arginine | 16.1 | 5.0 | 9.21 | 8.4 |
| Lysine | 7.1 | 7.8 | 6.5 | 6.2 |
| Proline | — | 5.1 | 4.4 | 3.0 |
| Leucine | 7.45 | 10.4 | 7.4 | 8.1 |
| Phenylalanine | 8.6 | 6.4 | 4.4 | 4.3 |
| Isoleucine | 8.7 | 6.1 | 3.9 | 4.1 |
| Histidine | — | 2.2 | 2.6 | 2.0 |
| Tyrosine | — | 5.7 | 3.6 | 2.7 |
| Methionine | 0.6 | 2.4 | 0.8 | 1.8 |
| Cysteine/cystine | — | 1.6 | 1.7 | 1.9 |
| Tryptophan | 1.2 | 1.4 | 1.2 | 1.3 |
| Ornithine | 0.4 | — | — | — |

The acylation reaction is known to persons skilled in the art. It is described for example in international application published under the number WO 98/09611. It is carried out indifferently on an amino acid or on a mixture of amino acids. The acylating agent generally consists of an activated derivative of a carboxylic acid of formula R₁—C(═O)—OH, in which R₁ is as defined above, such as a symmetric anhydride of this acid, the methyl ester of this acid, or an acid halide such as the acid chloride or the acid bromide. It may, also consist of a mixture of activated derivatives of carboxylic acids derived from natural oils or fats of animal or plant origin such as copra, palm kernel, palm, soya bean, rapeseed or maize oils, or beef tallow, spermaceti oil or herring oil. In the context of the present invention, mixtures of fatty acids derived from copra oil, palm kernel oil or spermaceti oil which contain a major dodecanoic acid fraction are preferably used:

| | | Palm kernel | Spermaceti |
|---|---|---|---|
| | Copra oil | oil (% by | oil (% by |
| | (% by weight) | weight) | weight) |
| Octanoic or | 6% to 9% | 3% to 10% | — |
| caprylic acid | | | |
| (C₈H₁₆O₂) | | | |
| Decanoic or | 6% to 10% | 3% to 14% | 1% to 3% |
| capric acid | | | |
| (C₁₀H₂₀O₂) | | | |
| Dodecanoic or | 44% to 51% | 37% to 52% | 14% to 38% |
| lauric acid | | | |
| (C₁₂H₂₄O₂) | | | |
| Tetradecanoic or | 13% to 18% | 7% to 17% | 12% to 14% |
| myristic acid | | | |
| (C₁₄H₂₈O₂) | | | |
| Hexadecanoic or | 8% to 10% | 2% to 9% | 8% to 10% |
| palmitic acid | | | |
| (C₁₆H₃₂O₂) | | | |
| Octadecanoic or | 1% to 3% | 1% to 3% | 1% to 3% |
| stearic acid | | | |
| (C₁₈H₃₆O₂) | | | |
| Octadecenoic or | 5.5% to 7.5% | 11% to 23% | 15% to 18% |
| oleic acid | | | |
| (C₁₈H₃₄O₂) | | | |
| Eicosenoic or | — | — | 5% to 8% |
| gadolic acid | | | |
| (C₂₀H₃₈O₂) | | | |
| Octadecadienoic | <2.5% | 1% to 3% | — |
| or linoleic acid | | | |
| (C₁₈H₃₂O₂) | | | |
| Other acids | <0.4% | <0.6% | 26% to 34% |

According to a particular aspect of the present invention, its subject is the use of N-cocoyl amino acids as slimming agent in a composition containing a cosmetically acceptable medium.

The expression N-cocoyl amino acids denotes a mixture of compounds of formula (I) obtained by acylation of an amino acid or of a mixture of amino acids with the activated derivatives of fatty acids derived from copra oil.

The subject of the invention is also a nontherapeutic method of treating the human body intended for slimming it, characterized in that a composition containing a cosmetically acceptable medium and an effective quantity of at least one compound of formula (I) as defined above, is applied to it.

The subject of the invention is also the use of at least one compound of formula (I), as defined above, for preparing a medicament with lipolytic activity, intended for inducing slimming of the human body.

In the compositions defined above, the compound of formula (I) is generally used in a quantity of between 0.01% and 10% of their weight, more particularly between 0.1% and 5% of their weight, and most particularly between 1% and 5% of their weight.

As the examples show, the compounds used in the cosmetic or therapeutic treatments defined above are characterized, unexpectedly, by a lipolytic activity greater than the compositions of the state of the art.

They are therefore in general appropriate for the slimming treatments of the human body.

The compositions used in the said treatments are generally provided in the form of dilute aqueous or aqueous-alcoholic solutions, in the form of simple or multiple emulsions, such as water-in-oil (W/O), oil-in-water (O/W) or water-in-oil-in-water (W/I/W) emulsions in which the oil is of a vegetable or mineral nature, or in powdered form. They may also be dispersed or impregnated onto textile or onto nonwoven materials such as wipes, paper serviettes or clothing.

The compositions used in the said treatments are administered to the subject in the conventional forms used in cosmetics and in pharmacy; this includes more particularly topical, oral or parenteral administrations.

In general, the compounds of formula (I) are combined with many types of adjuvants or active ingredients used in cosmetic formulations, such as fatty substances, organic solvents, thickeners, gelling agents, emollients, antioxidants, opacifiers, stabilizers, foaming agents, perfumes, emulsifiers, which are ionic or nonionic, fillers, sequestrants, chelators, preservatives, chemical screening agents or inorganic screening agents, essential oils, colouring matter, pigments, hydrophilic or lipophilic active agents, humectants, for example glycerin, preservatives, colorants, perfumes, cosmetic active agents, inorganic or organic sunscreens, inorganic fillers such as iron oxides, titanium oxides and talc, synthetic fillers such as nylons and poly(methyl methacrylate) which are crosslinked or otherwise, silicone elastomers, sericites or plant extracts or alternatively lipid vesicles or any other ingredient customarily used in cosmetics.

As examples of oils which may be combined with the compound of formula (I), there may be mentioned mineral oils such as paraffin oil, liquid paraffin, isoparaffins or white mineral oils, oils of animal origin, such as squalene or squalane, vegetable oils, such as sweet almond oil, copra oil, castor oil, jojoba oil, olive oil, rapeseed oil, groundnut oil, sunflower oil, wheat germ oil, maize germ oil, soya bean oil, cottonseed oil, lucerne oil, poppy seed oil, pumpkinseed oil, evening primrose oil, millet oil, barley oil, rye oil, safflower oil, candlenut oil, passionflower oil, hazelnut oil, palm oil, shea butter, apricot kernel oil, calophyllum oil, sysymbrium oil, avocado oil, calendula oil; ethoxylated vegetable oils; synthetic oils such as fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol. dicaprylate, esters derived from lanolic acid, such as isopropyl lanolate, isocetyl lanolate, monoglycerides, diglycerides and triglycerides of fatty acids such as glyceryl triheptanoate, alkyl benzoates, poly-alpha-olefins, polyolefins such as polyisobutene, synthetic isoalkanes such as isohexadecane, isododecane, perfluorinated oils and silicone oils. Among the latter, there may be mentioned more particularly dimethylpoly-siloxanes, methylphenylpolysiloxanes, silicones modified with amines, silicones modified with fatty acids, silicones modified with, alcohols, silicones modified with alcohols and fatty acids, silicones modified with polyether groups, epoxy-modified silicones, silicones modified with fluorinated groups, cyclic silicones and silicones modified with alkyl groups.

As other fatty substances which may be combined with this active agent, there may be mentioned fatty alcohols or fatty acids.

Among the thickening and/or emulsifying polymers used in the present invention are for example homopolymers or copolymers of acrylic acid or of acrylic acid derivatives, homopolymers or copolymers of acrylamide, homopolymers or copolymers of acrylamide derivatives, homopolymers or copolymers of acrylamidomethylpropane-sulphonic acid, vinyl monomer, trimethylaminoethyl-acrylate chloride, hydrocolloids of plant or biosynthetic origin, for example xanthan gum, karaya gum, carrageenans, alginates; silicates; cellulose and its derivatives; starch and its hydrophilic derivatives; polyurethanes. Among the polymers of the polyelectrolyte type which may be used in the production of a gelled aqueous phase capable of being used in the preparation of W/O emulsions containing the compounds of formula (I) which are the subject of the present invention, there are for example copolymers of acrylic acid and 2-methyl-[(1-oxo-2-propenyl)amino]-1-propanesulphonic acid (AMPS), copolymers of acrylamide and 2-methyl-[(1-oxo-2-propenyl)amino]-1-propane-sulphonic acid, copolymers of 2-methyl-[(1-oxo-2-propenyl)amino]-1-propanesulphonic acid and (2-hydroxyethyl) acrylate, homopolymer of 2-methyl-[(1-oxo-2-propenyl)amino]-1-propanesulphonic acid, homopolymer of acrylic acid, copolymers of acryloylethyltrimethylammonium chloride and acrylamide, copolymers of AMPS and vinylpyrrolidone, copolymers of acrylic acid and alkyl acrylates whose carbon chain comprises between ten and thirty carbon atoms, copolymers of AMPS and alkyl acrylates whose carbon chain comprises between ten and thirty carbon atoms. Such polymers are marketed respectively under the names SIMULGEL™ EG, SEPIGEL™ 305, SIMULGEL™ NS, SIMULGEL™ 800 and SIMULGEL™ A by the applicant.

Among the waxes which can be used in the present invention, there may be mentioned for example beeswax; carnauba wax, candelilla wax; ouricoury wax; Japan wax; cork fibre or sugarcane wax; paraffin waxes; lignite waxes; microcrystalline waxes; lanolin wax; ozokerite; polyethylene wax; hydrogenated oils; silicone waxes; vegetable waxes; fatty alcohols and fatty acids which are solid at room temperature; glycerides which are solid at room temperature.

Among the emulsifiers which can be used in the present invention, there may be mentioned for example fatty acids, ethoxylated fatty acids, fatty acid esters of sorbitol, ethoxylated fatty acid esters, polysorbates, polyglycerol esters, ethoxylated fatty alcohols, sucrose esters, alkyl polyglycosides, sulphated and phosphated fatty alcohols or mixtures of alkyl polyglycosides and fatty alcohols described in French Patent Applications 2 668 080, 2 734 496, 2 756 195, 2 762 317, 2 784 680, 2 784 904, 2 791 565, 2 790 977, 2 807 435 and 2 804 432.

As examples of an active ingredient which may be combined with the compound of formula (I) there may be mentioned compounds having a lightening or depigmenting action, such as for example arbutin, kojic acid, hydroquinone, ellagic acid, vitamin C, magnesium ascorbyl phosphate, extracts of polyphenols, grape extracts, pine extracts, wine extracts, olive extracts, marc extracts, N-acylated proteins, N-acylated peptides, N-acylated amino acids, partial hydrolysates of N-acylated proteins, amino acids, peptides, total hydrolysates of proteins, partial hydrolysates of proteins, polyols (for example glycerin or butylene glycol), urea, pyrrolidonecarboxylic acid or derivatives of this acid, glycyrrhetinic acid, alpha-bisabolol, sugars or sugar derivatives, polysaccharides or their derivatives, hydroxy acids, for example lactic acid, vitamins, vitamin derivatives such as retinol, vitamin E and its derivatives, minerals, enzymes, coenzymes such as Coenzyme Q10, hormones or hormone-like substances, soya bean extracts, for example Raffermine™, wheat extracts, for example Tensine™ or Gliadine™, vegetable extracts such as extracts rich in tannins, extracts rich in isoflavones or extracts rich in terpenes, extracts of fresh water or marine algae, essential waxes, bacterial extracts, minerals, lipids in general, lipids such as ceramides or phospholipids, active agents having a slimming action such as caffeine or its derivatives, active agents having an antimicrobial activity or a purifying action in relation to greasy skins such as LIPACIDE™ PVB, active agents having an energizing or stimulating property such as SEPITONIC™ M3 or Physiogenyl™, panthenol and its derivatives such as SEPICAP™ MP, antiageing active agents such as SEPILIFT™ DPHP, LIPACIDE™ PVB, SEPIVINOL™, SEPIVITAL™, moisturizing active agents such as SEPICALM™ S, SEPICALM™ VG and LIPACIDE™ DPHP, “antiphotoageing” antiageing active agents, active agents protecting the integrity of the dermoepidermal junction, active agents increasing the synthesis of the components of the extracellular matrix, active agents having a slimming, toning or draining activity such as caffeine, theophylline, cAMP, green tea, sage, ginko biloba, ivy, horse-chestnut, bamboo, ruscus, butcher's broom, centella asiatica, heather, meadowsweet, fucus, rosemary, willow, active agents creating a sensation of “heat” on the skin, such as activators of skin microcirculation (for example nicotinates) or products creating a sensation of “freshness” on the skin (for example menthol and its derivatives).

As sunscreen which may be incorporated into the composition according to the invention, there may be mentioned all those which appear in the amended cosmetics directive 76/768/EEC, annex VII.

The following experimental study illustrates the invention without, however, limiting it.

I) Evaluation in vitro of the Lipolytic Activity of the Compounds of Formula (I)

A—Assay of the Free Fatty Acids

(1)—Aim and Principle of the Method

The objective of the experiment is to demonstrate, in an in vitro model of isolated human adipocytes, the lipolytic activity of the compounds used. Hydrolysis of the triglycerides to nonesterified fatty acids and glycerol is called lipolysis. Triglycerides are stored in the adipocytes and constitute the fat reserve. For this reserve to diminish, which is the desired aim when slimming products are used, the triglycerides should be hydrolysed in the form of fatty acids, which in their case can be removed from the cell. Hydrolysis of the triglycerides calls into play a hormone-dependent lipase which must be phosphorylated in order to be active. The phosphorylation step uses a kinase and cAMP. An increase in the cAMP content of the adipocytes is necessary for promoting the activity of the lipase and therefore the lipolysis. The method described consists in incubating the products in the presence of human adipocytes in suspension, followed by measurement of the intracellular cAMP level.

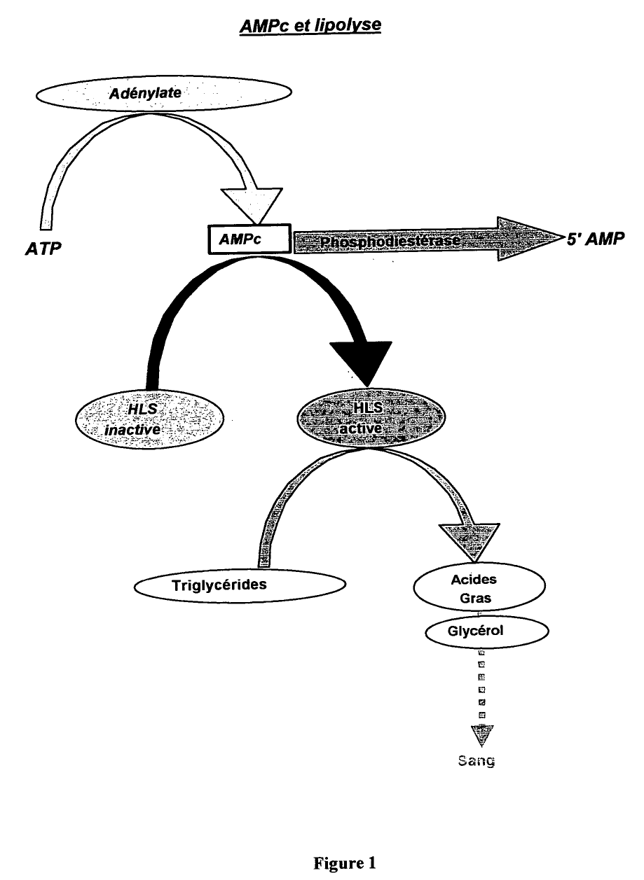

FIG. 1 illustrates this mechanism of action: the hormone-dependent lipase involved in the hydrolysis of the intraadipocyte triglycerides must be phosphorylated in order to be active. This phosphorylation calls into play cAMP. It is therefore important, in order to stimulate lipolysis, to increase the intracellular cAMP content either by increasing the production of cAMP by stimulating adenylate cyclase, or by reducing the degradation of cAMP by inhibiting phosphodiesterase (in particular PDE 3 in the adipocytes).

(2)—Experimental Protocol

(i) Cellular Model

The test is carried out using human adipocytes isolated and prepared as a cellular suspension. The adipocytes are isolated from the subcutaneous abdominal adipose tissue recovered during plastic surgery operations (abdominal plastic surgery operations) performed on women. The cells are isolated from fresh tissue. The adipose tissue is isolated and dissociated by the action of a collagenase (SIGMA™, 1 mg/ml, 30 minutes at 37° C., gentle stirring). Collagenase digests the connective tissue present in the adipose tissue. After digestion, the cells are filtered and washed in an appropriate culture medium containing MEM medium free of phenol red, free of glutamine (SIGMA)+2.2 mg/ml of sodium bicarbonate (GIBCO)+50 IU of penicillin (BIOWHITTAKER™)+50 μg/ml of streptomycin (BIOWHITTAKER™)+1% (v/v) of L-glutamine (BIOWHITTAKER™)+0.5% of lipid-free serum albumin (SIGMA™). The adipocyte suspension is used immediately after its preparation.

(ii) Incubation of the Products with the Adipocytes

The test products are diluted in the adipocyte culture medium. They are incubated with the cells in suspension for two hours at 37° C. (250 μl of product+250 μl of adipocyte suspension).

(3)—Evaluation of the Results

(i)—Concentration of Free Fatty Acids

After the incubation, the cell lysis is checked visually by the presence of a lipid layer at the surface of the cellular suspension. The supernatant media are collected. The free fatty acids are assayed by spectrophotometry using a commercial kit (NEFA™ C kit, WAKO), with reference to a fatty acid calibration series. The lypolytic activity of the products is evaluated relative to a control group incubated in the presence of adipocytes and in the absence of product. The reactivity of the adipocytes is systematically checked for the measurement of the lipolytic activity of the reference products, caffeine (1,3,7-trimethyl-xanthine) and theophylline (1,3-dimethyl-2,6-dihydroxy-purine). Five assays are performed for each of the test products which are as follows:

A composition A comprising:

| | |
|---|---|
| Lauroylglutamic acid: | 27% by weight |
| Lauroylaspartic acid: | 30% by weight |
| Lauroylglycine acid: | 6% by weight |
| Lauroyl alanine acid: | 8% by weight |
| Lauric acid: | 15% by weight |
| Water: | 10% by weight |
| Sodium chloride: | 1% by weight |
| Propylene glycol: | 3% by weight |

A composition A′ comprising between 96% and 97% by weight of composition A as defined above and from 3% to 4% by weight of magnesium and potassium aspartate;

A composition B comprising from about 30% to 40% by weight of N-cocoyl amino acid active material;

A composition C which is a mixture comprising about 75% by weight of composition B) and about 25% by weight of PECOSIL™ SPP 50 (100% of potassium dimethicone copolyol panthenyl phosphate); and

N-lauryl sarcosinate

The results of the trials, expressed by the arithmetic means of the five assays carried out for each of the products, are presented in the following table:

| | | | Lipolytic |
|---|---|---|---|
| | | | activity |
| | | Free fatty | (compared |
| | Incubation | acid | with the |
| | concentration | concentration | control = |
| | (wt % DE) | (μM) | 100) |
| Control | — | 11.54 ± 2.71 | 100 |
| Caffeine | 0.0019 | 15.10 ± 4.11 | 131 |
| Theophylline | 0.0019 | 16.53 ± 4.23 | 143 |
| Composition A | 0.0001 | 21.19 ± 0.85 | 184 |
| Composition A | 0.0010 | 32.67 ± 3.14 | 283 |
| Composition A′ | 0.0001 | 26.37 ± 1.89 | 229 |
| Composition A′ | 0.0010 | 32.25 ± 0.39 | 279 |
| Composition B | 0.0001 | 24.64 ± 0.21 | 213 |
| Composition B | 0.0010 | 29.46 ± 0.56 | 241 |
| Composition C | 0.0001 | 23.66 ± 1.33 | 205 |
| Composition C | 0.0010 | 23.60 ± 0.59 | 206 |
| N-Lauroylsarcosine | 0.0001 | 26.32 ± 0.68 | 228 |
| N-Lauroylsarcosine | 0.0010 | 28.82 ± 6.11 | 250 |

DE: dry extract

These results show that while the slimming compositions of the state of the art (caffeine and theophylline) act on lipolysis with a multiplication factor of 1.3 to 1.4 relative to the control, those according to the invention, that is to say comprising at least one compound of formula (I) as defined above, act with a factor of 1.8 to 2.8 at lower concentrations.

This assay is repeated by carrying out new assays at different concentrations. The results are presented in the following table:

| | | Assay of the |
|---|---|---|
| | | nonesterified |
| | | fatty acids in |
| | Incubation | the supernatant |
| | concentration | (% relative to |
| | (wt % DE) | the control) |
| Control | — | 100 |
| Caffeine | 0.0005 | 200 |
| Caffeine | 0.0025 | 240 |
| Composition A | 0.0005 | 155 |
| Composition A | 0.0025 | 190 |

B—Assay of Intracellular cAMP

Adipocytes in suspension are drained by aspirating the supernatant medium. The intracellular content of cAMP is assayed by an EIA technique. with the aid of a kit (AMERSHAM™, RPN225). The assay is performed against a cAMP calibration series. The effect of the products on the cAMP content is evaluated in relation to a control group incubated in the presence of adipocytes and in the absence of product. The reactivity of the adipocytes is systematically checked by measuring the effect of a reference product, caffeine.

The results of the trials, expressed as intracellular content of cAMP relative to the control, are presented in the following table:

| | | Intracellular |
|---|---|---|
| | | content of |
| | Incubation | cAMP (% |
| | concentration | relative to |
| | (wt % DE) | the control) |
| Control | — | 100 |
| Caffeine | 0.0005 | 200 |
| Caffeine | 0.0025 | 450 |
| Composition A | 0.0005 | 220 |
| Composition A | 0.0025 | 250 |

C—Measurement of the Activity of Phosphodiesterase Type 3

(1)—Principle of the Method

Phosphodiesterases (PDE) are enzymes which degrade cAMP to 5′-AMP. Phosphodiesterase type 3 (PDE 3) is present in human adipocytes. The inhibition of this enzyme makes it possible to increase the intracellular content of cAMP and therefore to activate lipolysis. The test is based on a biochemical evaluation using a purified enzyme.

(2)—Experimental Protocol—Results

Phosphodiesterase type 3, isolated from human platelets, is incubated in the presence of tritium-labelled cAMP, an excess of nonlabelled cAMP and in the presence of the test product or with no product (control) . The incubation is carried out at 30° C. for 30 minutes. At the end of the incubation, the quantity of tritium-labelled 5′-AMP produced is quantified with the aid of a radioactivity counter. The reactivity of the enzymatic system is systematically checked by measuring the effect of a reference product, milrinone. The results are presented in the following table as a percentage inhibition of the activity of phosphodiesterase 3 (PDE 3).

| | | Percentage |
|---|---|---|
| | Incubation | inhibition |
| | concentration | of PDE 3 |
| | (in % DE) | activity: |
| Caffeine | 0.001 | 20% |
| Caffeine | 0.01 | 70% |
| Composition A | 0.001 | 0% |
| Composition A | 0.01 | 60% |

D—Measurement of the Activity of Lipase

(1)—Principle of the Method

Lipase is an enzyme which degrades triglycerides to fatty acids and glycerol according to the following reaction scheme:

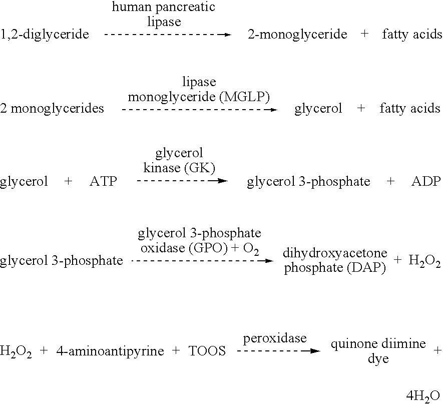

TOOS: sodium N-ethyl-N-(2-hydroxy-3-sulphopropyl)-3-toluidine

Inside the adipocytes, the free fatty acids can thus be removed from storage. The objective of this test is to demonstrate a lipase stimulating effect. It is based on an enzymatic test, using a purified enzyme.

(2)—Experimental Protocol—Results

A human pancreatic lipase (SIGMA, LIPASE-PS™ kit) is incubated in the presence of a diglyceride and a reaction system to produce a coloured quinone diimine which can be assayed by spectrophotometry (550 nm). The measurement of the absorbance at 550 nm is directly proportional to the activity of the lipase.

All the results are provided in the LIPASE-PS™ kit (SIGMA) . The product is incubated with the reaction mixture for 8 minutes at 37° C.

The reaction system used is composed of:

1,2-Diglyceride: 1.1 mM

TOOS: 2.0 mM

ATP: 0.66 mM

MGLP (microbial): 860 U/l

GK (microbial): 1340 U/l

GPO (microbial): 40 000 U/l

Peroxidase (horseradish): 1340 U/l

Colipase (pig): 40 000 U/l

The results are presented in the following table as a percentage activation of the lipase relative to the control.

| | Incubation | Percentage |
|---|---|---|
| | concentration | activation |
| | (in % DE) | of the lipase |
| Caffeine | 0.01 | 0% |
| Caffeine | 0.1 | 10% |
| Composition A | 0.01 | 25% |
| Composition A | 0.1 | 140% |

E—Measurement of the Activity of Lipoprotein Lipase

(1)—Principle of the Method

Lipoprotein lipase (LPL) is an enzyme found in the extracellular compartment of adipocytes. It brings about the hydrolysis of the triglycerides contained in the chylomicrons to fatty acids and glycerol. The latter can thus penetrate into the adipocytes to be stored therein. The objective of this test is to demonstrate a lipoprotein lipase inhibiting effect. It is based on an enzymatic test using a purified enzyme.

(2)—Experimental Protocol; Results

A lipoprotein lipase (SIGMA, pseudomonas species) is incubated in the presence of a substrate, p-nitrophenyl butyrate (PNPB), which absorbs light at 400 nm after hydrolysis.

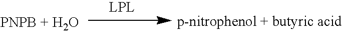

The enzyme, at 20 U/ml, is incubated in the presence of the test product and PNPB, at 0.9 mM. The kinetics of appearance of p-nitrophenol is performed at 37° C. for 10 minutes. It is monitored by measuring the absorbance at 400 nm.

The results are presented in the following table, as a percentage inhibition of the lipoprotein lipase activity relative to the control.

| | Incubation | Percentage |
|---|---|---|
| | concentration | inhibition |
| | (in % DE) | of LPL |
| Caffeine | 0.01 | 0% |
| Caffeine | 0.1 | 0% |
| Composition A | 0.01 | 5% |
| Composition A | 0.1 | 40% |

F—Measurement of the Activity of the Metalloproteinases MMP-2 and MMP-9

(1)—Principle of the Method

The metalloproteinases MMP-2 and MMP-9 are found in human adipocytes. There they play a role in the differentiation of the preadipocytes to adipoctyes and therefore in the adipose tissue storage capacity.

The objective of this test is to evaluate the inhibitory capacity of a product on the enzymatic activity of MMP-2 and MMP-9. This test is based on an enzymatic reaction between purified MMP-2 or MMP-9 and a specific substrate containing a chromophore. The degradation of the chromogenic substrate by the enzyme leads to the production of fluorescence monitored by fluorimetry.

(2)—Experimental Protocol

(i)—A human recombinant MMP-9 (CALBIOCHEM, PF024) is incubated, at 0.05 μg/ml, in the presence of the product and the chromogenic substrate, DQ-gelatin conjugated with fluorescein (MOLECULAR PROBE, D12054) at 10 μg/ml. The reaction mixture is incubated overnight at room temperature, protected from light. The quantity of DQ-gelatin degraded is measured by fluorescence (Ex: 355 nm; Em: 460 nm).

(ii)—A human recombinant MMP-2 (CALBIOCHEM, PF023) is incubated, at 0.1 μg/ml, in the presence of the product and the chromogenic substrate, DQ-gelatin conjugated with fluorescein (MOLECULAR PROBE, D12054) at 10 μg/ml. The reaction mixture is incubated overnight at room temperature, protected from light. The quantity of DQ-gelatin degraded is measured by fluorescence (Ex: 355 nm; Em: 460 nm).

The reactivity of the enzymatic system is systematically monitored for the measurement of the effect of a metalloproteinase inhibitor, 1,10-phenanthroline. The results are presented in the following table, as a percentage inhibition of the enzymatic activity of MMP-2 and MMP-9 relative to the control.

| | Incubation | MMP-2 | MMP-9 |
|---|---|---|---|
| | concentration | inhibiting | inhibiting |
| | (in % DE) | effect | effect |
| 1,10-Phenanthroline | 0.0018 | 25% | 75% |
| Caffeine | 0.1 | 0% | 0% |
| Composition A | 0.1 | 40% | 35% |

G—Use of the α₂- and β-adrenergic Receptors (for the Lipolytic Activity)

(1)—Principle of the Method

It involves determining, in an “in vitro” model of isolated human adipocytes, the involvement of the α₂- and/or β-adrenergic receptors on the lipolytic activity of the test product. Lipolysis is indeed under the control of the adrenergic receptors found at the surface of the adipocytes. Two types of receptors coexist:

the β-adrenergic receptors, whose stimulation induces lipolysis

the α₂-adrenergic receptors, whose stimulation inhibits lipolysis.

A lipolytic product can call into play these receptors either by stimulating the β-adrenergic receptors (agonist), or by inhibiting the α₂-adrenergic receptors (antagonist). The method described consists in incubating the product in the presence of human adipocytes in suspension, followed by the measurement of the lipolytic activity by assaying fatty acids. The adipocytes are incubated in the presence of pharmacological agents, α₂-adrenergic agonists or β-adrenergic antagonists, known to inhibit lipolysis. The trial consists in studying the effect of the product on the inhibition of lipolysis induced by these pharmacological agents.

(2)—Experimental Protocol—Results

(i)—Cellular Model

The test is carried out using human adipocytes isolated and prepared as a cellular suspension. The adipocytes are isolated from the subcutaneous abdominal adipose tissue recovered during plastic surgery operations (abdominal plastic surgery operations) performed on women. The cells are isolated from fresh tissue. To isolate the cells, the adipose tissue is isolated and dissociated by the action of a collagenase (SIGMA™, 1 mg/ml, 30 minutes at 37° C., gentle stirring). Collagenase digests the connective tissue present in the adipose tissue. After digestion, the cells are filtered and washed in an appropriate culture medium containing MEM medium free of phenol red, free of glutamine (SIGMA)+2.2 mg/ml of sodium bicarbonate (GIBCO)+50 IU of penicillin (BIOWHITTAKER™)+50 μg/ml of streptomycin (BIOWHITTAKER™)+1% (v/v) of L-glutamine (BIOWHITTAKER™)+0.5% of lipid-free serum albumin (SIGMA™) The adipocyte suspension is used immediately after its preparation.

(ii) Incubation of the Products with the Adipocytes

The product is diluted in the adipocyte culture medium.

The product is incubated with the cells in suspension for two hours at 37° C. (250 μl of product+250 μl of adipocyte suspension).

The product is incubated under three experimental conditions:

alone,

in the presence of an α₂-adrenergic agonist: brimonidine at 10⁻⁹ M (concentration previously determined to inhibit basal lipolysis by about 50%), and

in the presence of a β-adrenergic antagonist: propranolol at 10⁻⁷ M (concentration previously determined to inhibit basal lipolysis by about 50%).

(iii)—Evaluation Parameters

At the end of the incubation, the cellular lysis is checked macroscopically for the absence of a lipid layer at the surface of the cells.

The supernatant media are collected. The nonesterified fatty acids are assayed by spectrophotometry with the aid of a commercial kit in these supernatant media (NEFA C kit, WAKO). The assay is performed against a fatty acid calibration series.

The reactivity of the α₂-adrenergic and β-adrenergic receptors of the adipocytes is systematically checked for the measurement of the lifting of the inhibition induced by the pharmacological agents, namely:

for the inhibition induced by the α₂-adrenergic agonist, by the use of phentolamine which is an α₂-antagonist,

for the inhibition induced by the β-adrenergic antagonist, by the use of a β-adrenergic agonist, isoprenaline.

The results are presented in the following tables as nonesterified fatty acids in the supernatant (% relative to the control)

| | | Assay of the |
|---|---|---|
| | | nonesterified |
| | | fatty acids in |
| | | the supernatant |
| | Incubation | (% relative to |
| | concentration | the control) |
| Control | — | 100% |
| Brimonidine | 10⁻⁹ M | 0% |
| Composition A | 0.002% DE + | 100% |
| | brimonidine 10⁻⁹ M | |
| Phentolamine | 10⁻⁷ M + | 79% |
| | brimonidine 10⁻⁹ M | |

The composition A according to the invention therefore has an inhibitory effect on the antilipolytic effect of an α₂-adrenergic agonist.

| | | Assay of the |
|---|---|---|
| | | nonesterified |
| | | fatty acids in |
| | | the supernatant |
| | Incubation | (% relative to |
| | concentration | the control) |
| Control | — | 100% |
| Propanolol | 10⁻⁷ M | 86% |
| Composition A | 0.002% DE + | 115% |
| | propanolol 10⁻⁷ M | |
| Isoprenaline | 10⁻⁵ M + | 170% |
| | propanolol 10⁻⁷ M | |

The composition A according to the invention therefore has an inhibitory effect on the antilipolytic effect of a β-adrenergic antagonist.

H—Measurement in vitro of the Production of Hyaluronic Acid

(1)—Principle of the Method

Glycosaminoglycans (GAGs) are the major proteoglycans of the dermis and play an essential role in maintaining the integrity of the skin. Hyaluronic acid is a major nonsulphated GAG which plays an essential role in the hydration of the skin by its capacity to fix up to 1000 times its weight as water. The effect of the products on the hyaluronic acid level was evaluated by a technique in vitro.

(2)—Experimental Protocol—Plan of Study—Results

The hyaluronic acid level is measured in cultures of normal human dermal fibroblasts. The cells are incubated for 5 days in the presence of the products solubilized in the incubation medium. At the end of this incubation, the extracellular media in which the hyaluronic acid is secreted are collected. The hyaluronic acid is stained with a specific stain, STAINS ALL ((1-ethyl-2-[3-(1-ethylnaphtho-[1,2-d]thiazolin-2-ylidene)-2-methylpropenyl]naphtho-[1,2-d]thiazolium bromide, SIGMA), which interacts with it to produce a change in the absorption spectrum between 620 and 660 nm; the measurement is made by spectrophotometry. A hyaluronic acid series is prepared in parallel.

Plan of study:

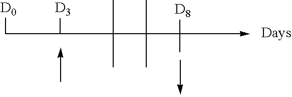

DO: inoculation of the fibroblasts (24-well culture plates, 15 300 cells/well,

D3: incubation of the test products, diluted in the fibroblast incubation medium

D8: collection of the fibroblast incubation media, assay of hyaluronic acid

Evaluation of the effects: After 5 days of incubation in the presence of the products, the incubation media are collected and incubated in the presence of STAINS ALL. The calorimetric reaction is developed by adding water. The quantification is made by spectrophotometry at 630 nm. A hyaluronic acid series is prepared in parallel. The results are expressed as extracellular hyaluronic acid relative to the control.

| | |
|---|---|
| | Extracellular |
| | hyaluronic |
| | acid produced |
| Incubation | (% relative to |
| concentration | the control) |

| Control | — | | 100% |
|---|---|---|---|
| EGF | 10 | ng/ml | 133% |
| Composition A | 0.001% | DE | 130% |
| Caffeine | 0.001% | DE | 131% |

I—Measurement of the Activity of Hyaluronidase

(1)—Principle of the Method

The objective of this test is to measure the activity of hyaluronidase. This enzyme, which is naturally found in the skin, degrades hyaluronic acid. The study is carried out in vitro on the purified enzyme.

(2)—Experimental Protocol—Results

The hyaluronidase (SIGMA), purified from bovine testicles, is incubated at 0.01 U/ml in the presence of hyaluronic acid at 0.02 mg/ml, with or without test product. The incubation is carried out for 60 minutes at 37° C.

Evaluation of the effects: After the incubation, the hyaluronic acid not degraded by the enzyme is precipitated by a 24 mM sodium acetate/79. mM acetic acid mixture (10 minutes, room temperature). The precipitate is measured by spectrophotometry at 590 nm.

The effect of the test products is compared to that observed in the presence of dipotassium glycyrrhizinate, a reference hyaluronidase inhibitor.

| | | Percentage |
|---|---|---|
| | Incubation | inhibiton |
| | concentration | of the PDE 3 |
| | (in % DE) | activity: |
| Di K glycyrrhizinate | 0.001 | 20% |
| Di K glycyrrhizinate | 0.1 | 45% |
| Caffeine | 0.001 | 15% |
| Caffeine | 0.01 | 20% |
| Composition A | 0.001 | 20% |
| Composition A | 0.01 | 50% |

II—Examples of Cosmetic Formulations

In the following examples, the proportions are expressed as percentages by weight.

| Example 1: Slimming body milk | |
|---|---|
| MONTANOV ™ L | 3.00% |
| Phytosqualane | 8.00% |
| Sweet almond oil | 2.00% |
| Water | qsp 100% |
| SEPIGEL ™ 501 | 1.50% |
| Composition A′ | 3.00% |
| SEPICIDE ™ CI | 0.20% |
| SEPICIDE ™ HB | 0.30% |
| Perfume | 0.30% |
| Example 2: Anti-sagging cream (oval target of the face) | |
| MONTANOV ™ 202 | 3.50% |
| MONTANOV ™ 14 | 1.00% |
| SEPILIFT ™ DPHP | 1.00% |
| LANOL ™ 1688 | 15.00% |
| Wheat germ oil | 5.00% |
| Water | qsp 100% |
| SIMULGEL ™ EG | 1.30% |
| Composition A′ | 2.00% |
| SEPICIDE ™ CI | 0.20% |
| SEPICIDE ™ HB | 0.30% |
| Perfume | 0.10% |
| Example 3: Anti-plumpness spray | |
| MONTANE ™ 60 | 3.30% |
| MONTANOX ™ 60 | 1.70% |
| Caprylic/capric triglycerides | 6.00% |
| Isohexadecane | 5.00% |
| Magnesium Aluminium Silicate | 1.50% |
| Water | qsp 100% |
| SIMULGEL ™ EG | 1.00% |
| Composition A′ | 2.00% |
| Centalla asiatica/hydrocotyle extract | 1.00% |
| SEPICIDE ™ CI | 0.20% |
| SEPICIDE ™ HB | 0.30% |
| Perfume | 0.40% |
| Water | qsp 100% |
| Example 4: Refreshing slimming gel | |
| SEPIGEL ™ 305 | 3.50% |
| Hydroxyethylcellulose | 1.00% |
| Caffeine | 5.00% |
| Menthol | 0.30% |
| Ethanol | 50.00% |
| Composition A′ | 3.00% |
| SEPICIDE ™ LD | 1.00% |
| Perfume | 0.20% |
| Water | qsp 100% |
| Example 5: Slimming body fluid | |
| SIMULGEL ™ NS | 2.50% |
| Xanthan gum | 0.20% |
| LANOL ™ 99 | 5.00% |
| Composition A′ | 2.00% |
| Ginkgo biloba extract | 2.00% |
| Cola extract | 1.00% |
| Ginseng extract | 0.50% |
| SEPICIDE ™ HB | 1.50% |
| Perfume | 0.10% |
| Water | qsp 100% |
| Example 6: Toning revitalizing lotion | |
| intended to be impregnated into body wipes | |
| Composition A′ | 1.50% |
| Glycerin | 5.00% |
| Ethanol | 5.00% |
| Ruscus extract | 3.00% |
| SEPITONIC ™ M3 | 1.00% |
| SEPICIDE ™ CI | 0.20% |
| SEPICIDE ™ HB | 0.30% |
| Water | qsp 100% |
| Example 7: Slimming shower gel | |
| MONTALINE ™ C40 | 8.00% |
| PROTEOL ™ OAT | 5.00% |
| Sodium lauryl sulphate | 9.00% |
| Composition A′ | 3.00% |
| Green tea extract | 1.00% |
| KATHON ™ CG | 0.80 |
| Green colorant | qs |
| Green tea perfume | 1.00% |
| Lactic acid | qs |
| | pH = 6.5 |
| Water | qsp 100% |
| Example 8: Biphasic disinfiltrating massage | |
| Arabic coffee oil | 1.00% |
| LANOL ™ 189 | 20.00% |
| LANOL ™ 99 | 10.00% |
| Borage oil | 2.00% |
| Perfume | 0.10% |
| Compositon A′ | 3.00% |
| Glycerin | 3.00% |
| Ethanol | 10.00% |
| Blue colorant | qs |
| Water | qsp 100% |

The definitions of the commercial products used in the examples are the following:

SEPILIFT™ DPHP: (INCI name: Dipalmitoyl hydroxyproline), marketed by the company SEPPIC;

SEPICIDE™ CI: Imidazoline urea (preservative), marketed by the company SEPPIC;

SEPICIDE™ HB: Mixture of phenoxyethanol, methylparaben, ethylparaben, propylparaben and butylparaben (preservative), marketed by the company SEPPIC;

SEPICIDE™ LD: Phenoxyethanol marketed by the company SEPPIC;

KATHON™ CG: (INCI name: methyl isothiazolinone/Methyl chloroisothiazolinone);

MONTANE™ 60: Sorbitan stearate;

MONTANOX™ 60: Polysorbate 60;

SIMULGEL™ EG: Self-reversible invert latex of copolymer such as those described in international publication WO 99/36445 (INCI name: Sodium acrylate/Sodium acryloyldimethyl taurate copolymer and Isohexadecane and Polysorbate 80) marketed by the company SEPPIC; SIMULGEL™ NS: Self-reversible invert latex of copolymer such as those described in international publication WO 99/36445 (INCI name: hydroxyethylacrylate/Sodium acryloyldimethyl taurate copolymer and squalane and Polysorbate 60) marketed by the company SEPPIC;

SEPIGEL™ 305: Self-reversible invert latex (INCI name: Polyacrylamide/C13-14 Isoparaffin/Laureth-7);

SEPIGEL™ 501: Self-reversible invert latex (INCI name: C13-14 Isoparaffin/Mineral Oil/Sodium polyacrylate/Polyacrylamide/Polysorbate 85) marketed by the company SEPPIC;

LANOL™ 99: Isononyl isononanoate marketed by the company SEPPIC;

LANOL™ 189: Isostearyl isononanoate

LANOL™ 1688: Cetearyl ethyl hexanoate marketed by the company SEPPIC;

SEPITONIC™ M3: Mixture of magnesium aspartate, copper gluconate and zinc gluconate marketed by the company SEPPIC;

MONTALINE™ C40: Cocamidopropyl betainamide MEA chloride

PROTEOL™ OAT: N-lauroyl-containing oat amino acids;

MONTANOV™ 14: Myristyl alcohol/Myristyl glucoside;

MONTANOV™ L: Emulsifying agent based on a C14-C22 alcohol and a C12-C20 alkyl polyglucoside such as those described in European Patent Application EP 0 995 487;

MONTANOV™ 202 is an emulsifying agent based on arachidyl alcohol, behenyl alcohol and arachidyl polyglucoside.

1. Use of a compound of formula (I): 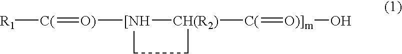
in which R₁ represents a linear or branched, saturated or unsaturated, aliphatic hydrocarbon radical comprising 11 carbon atoms, R₂ represents the characterizing chain of an amino acid and m is between 1 and 50, or of a mixture of the said compounds of formula (I), as a slimming active agent, in a composition containing a cosmetically acceptable medium.

2. Use as defined in claim 1, for which in formula (I), R₂ represents the characterizing chain of an amino acid chosen from glycine, alanine, serine, aspartic acid, glutamic acid, valine, threonine, arginine, lysine, proline, leucine, phenylalanine, isoleucine, histidine, tyrosine, tryptophan, asparagine, glutamine, cysteine, cystine, methionine, hydroxyproline, hydroxylysine, sarcosine or ornithine.

3. Use as defined in claim 2, for which in formula (I), R₂ represents the characterizing chain of glycine, alanine, aspartic acid, glutamic acid or sarcosine.

4. Use as defined in one of claims 1 to 3, in which m is a decimal number between 1 and 10 and is preferably less than 5.

5. Use as defined in claim 4, in which m is a decimal number of less than or equal to 2 and is more particularly less than or equal to 1.4.

6. Use as defined in one of claims 1 to 5, in which m is equal to 1.

7. Use as defined in one of claims 1 to 6, of N-cocoyl amino acids.

8. Nontherapeutic method of treating the human body intended for slimming it, characterized in that a composition containing a cosmetically acceptable medium and an effective quantity of at least one compound of formula (I) as defined in one of claims 1 to 7, is applied to it.

9. Use of at least one compound of formula (I), as defined in one of claims 1 to 7, for preparing a medicament with lipolytic activity, intended for inducing slimming of the human body.
